(19) United States
(12) Patent Application Publication
(10) Pub. No.: US 2022/0238205 A1
(43) Pub. Date: Jul. 28, 2022

(54) SYSTEMS AND METHODS TO PROVIDE A DIGITAL EXPERIENCE ADAPTED BASED ON A SUBJECT SELECTION TO EFFECT PARTICULAR ATTRIBUTES

(71) Applicant: Solsten, Inc., St. Paul, MN (US)

(72) Inventors: Joseph Jack Schaeppi, Maple Grove, MN (US); Lynn Danielle Francoise Bergmann, Berlin (DE); Lloyd William West, Berln (DE); Jonna Maarit Koivisto, Berlin (DE)

(21) Appl. No.: 17/160,190

(22) Filed: Jan. 27, 2021

(51) Int. Cl. G16H 20/70; G16H 50/20

(52) U.S. Cl. CPC G16H 20/70; G16H 50/20

(57) ABSTRACT

Systems and methods to provide a digital experience adapted based on a subject selection to effect particular attributes are disclosed. Exemplary implementations may: obtain subject information related to subjects; effectuate presentation of an outcome interface that enables selection and/or entry of one or more psychological outcomes; receive indication of the psychological outcomes selected and/or entered by the subjects; identify, based on the subject information and the one or more psychological outcomes, therapeutic content from content that individual online platforms present; receive interaction information from the individual online platforms; update in an ongoing manner the psychological profiles of the individual subjects based on the interaction information of the individual subjects; determine, based on the interaction information and the psychological profiles, a progress metric value to a progress metric indicating progress towards achieving the psychological outcomes selected; and adjust, based on the progress metric value, the therapeutic content.

### FIELD OF THE DISCLOSURE

The present disclosure relates to systems and methods to provide a digital experience adapted based on a subject selection to effect particular attributes.

### BACKGROUND

As use of online platforms and mobile applications increase, individuals may seek counsel in mental health and/or other circumstances virtually while still guided by caregivers. However, interactions of the individuals with online platforms that are external to the interactions between the individuals and the caregivers may affect, positively or negatively, achieving changes in attributes or psychological outcomes by the individuals.

### SUMMARY

One aspect of the present disclosure relates to a system configured to adapt digital experiences for a subject based on a selection or input of psychological outcomes by the subject. The system may obtain subject information that characterizes a psychological profile of the subject. The subject may be presented an outcome interface that enables the subject to select and/or enter one or more psychological outcomes to achieve. Psychological outcomes may correspond to changes in parameter value to one or more psychological parameters that are defined by the psychological profile. Based on the psychological outcomes selected, therapeutic content for individual online platforms may be identified for presentation to the subject during interaction with the online platforms. Interaction information that characterized engagement with the therapeutic content by the subject may be received from the online platforms. The psychological profile may be updated based on the interaction information and may be a basis to determine a progress metric value. The progress metric value may indicate progress towards achieving the psychological outcomes and be a basis for adjusting the therapeutic content for the online platforms. Thus, as the subject interacts with the online platforms, the online platforms are facilitating attaining the psychological outcomes.

One aspect of the present disclosure relates to a system configured to provide a digital experience adapted based on a subject selection to effect particular attributes. The system may include one or more hardware processors configured by machine-readable instructions, an outcome interface, and/or other components. Machine-readable instructions may include one or more instruction components. The instruction components may include one or more of interface presentation component, content identifying component, profile update component, progress component, and/or other instruction components.

The content identifying component may be configured to obtain subject information related to subjects and/or other information. The subject information may characterize psychological profiles of the individual subjects. The psychological profile may be defined by parameter values of psychological parameters.

The interface presentation component may be configured to effectuate presentation the outcome interface. The outcome interface may be presented via client computing platforms associated with the subjects. The outcome interface may enable selection and/or entry of one or more psychological outcomes from a catalog of psychological outcomes by the subjects. A given psychological outcome may be associated with changes in parameter value for one or more psychological parameters that correspond to the given psychological outcome. As such, the given psychological outcome may be achieved by achieving the associated changes in parameter value for the corresponding one or more psychological parameters.

The content identifying component may be configured to receive, from the client computing platforms, indication of the psychological outcomes selected and/or entered by the subjects. In some implementations, the subjects may select one of the psychological outcomes from the catalog or a plurality of the psychological outcomes from the catalog. In some implementations, the subjects may enter a psychological outcome that is not included in the catalog.

The content identifying component may be configured to identify therapeutic content from content that individual online platforms present. The therapeutic content may be identified based on the subject information and the one or more psychological outcomes selected and/or entered. The therapeutic content may be presented to the subjects via the individual online platforms and may facilitate achievement of the one or more psychological outcomes.

The profile update component may be configured to receive interaction information from the individual online platforms. The interaction information may characterize engagement by the subjects with the therapeutic content.

The profile update component may be configured to update in an ongoing manner the psychological profiles of the individual subjects based on the interaction information of the individual subjects.

The progress component may be configured to determine, in an ongoing manner, a progress metric value to a progress metric that indicates progress towards achieving the psychological outcomes selected. The determination of the progress metric value may be based on the interaction information and the psychological profiles. The progress component may be configured to adjust, in an ongoing manner, the therapeutic content presented to the subjects. Adjusting the therapeutic content may be based on the progress metric value.

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system configured to provide a digital experience adapted based on a subject selection to effect particular attributes, in accordance with one or more implementations.

FIG. 2 illustrates a method to provide a digital experience adapted based on a subject selection to effect particular attributes, in accordance with one or more implementations.

FIG. 3 illustrates an example implementation of the system configured to provide a digital experience adapted based on a subject selection to effect particular attributes, in accordance with one or more implementations.

### DETAILED DESCRIPTION

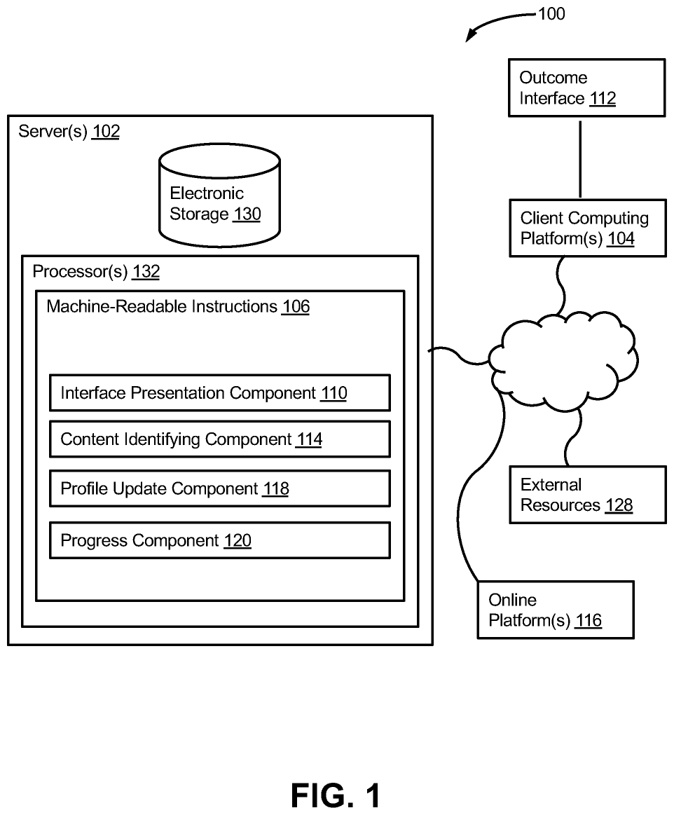

FIG. 1 illustrates a system 100 configured to provide a digital experience adapted based on a subject selection to effect particular attributes, in accordance with one or more implementations. As used herein, “online platform” or variations thereof may include or provide a digital experience. A digital experience may include one or more of a game (e.g., online via web browser, via desktop application, via mobile application, via game console, etc.), a website (e.g., retailers), a mobile application, a desktop application, a social network platform, a service provider (e.g., E-mail provider), and/or other digital experiences. Such digital experiences may collect similar information related to its subjects and provide some of the same interactions or similar interactions between individual subjects, between the individual subjects and virtual content, and/or other interactions. Virtual content (or pieces thereof) may include one or more of game content, social media content, publicity content, and/or other virtual content. The game content may include one or more of a player-controlled character, a non-player-controlled character, a task, a quest, an assignment, a mission, a level, a chapter, a mini-game, a virtual item, a virtual resource (e.g., weapon, tool), an in-game power, an in-game skill, an in-game technology, and/or other pieces of game content. By way of non-limiting example, virtual items may include one or more of clothing, pets, transportation units (e.g., aircrafts, motor vehicles, watercrafts, etc.), units, buildings, and/or other virtual items. The social media content and the publicity content may include one or more of an image, a video, a description, a financial component (e.g., for purchasing, selling, trading, donating, etc.), and/or other content. Online platforms 116 may be accessed by the subjects via client computing platform(s) 104.

In some implementations, system 100 may include one or more servers 102, an outcome interface 112, online platforms 116, and/or other components. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Subjects may access system 100 via client computing platform(s) 104.

Server(s) 102 may be configured by machine-readable instructions 106. Machine-readable instructions 106 may include one or more instruction components. The instruction components may include computer program components. The instruction components may include one or more of interface presentation component 110, content identifying component 114, profile update component 118, progress component 120, and/or other instruction components.

Content identifying component 114 may be configured to obtain subject information related to subjects. The subject information may characterize psychological profiles of the individual subjects. In some implementations, the subject information may include identifying information. By way of non-limiting example, the identifying information may include a name, a gender, an age, a subject name, an email address, a phone number, and/or other identifying information. In some implementations, the identifying information may be associated with the psychological profiles to identify the subjects. In some implementations, the psychological profiles may include the identifying information to identify the subjects. The psychological profile may be defined by parameter values of psychological parameters. The parameter values of the psychological parameters may characterize mental health and/or other information (e.g., medical history, past adversities, etc.) of a given subject. By way of non-limiting illustration, a first psychological profile may characterize the mental health and/or other information of a first subject.

The parameter values may define, by way of non-limiting example, achievement motivation, motivation, personality inventory, cultural values, competitiveness, positive and negative affect before, during, and/or after engagement with the online game (i.e., emotions), communication style, personal values, daily routines/activities, life/gaming pain points, life/gaming hopes and aspirations, wellbeing, subject experience, gaming/experience using time, subscription behavior, affinity information, personality, emotional style, goal orientation, goal commitment, ego and task orientation, relatedness, sense of community, social influence, social identity, group identification, we-identity, quality of life, satisfaction with life, work-related quality of life, mindfulness, happiness, emotional intelligence, self-awareness/internal awareness, external awareness, connectedness to nature, social connectedness, social bonding, perceived stress, depression, anxiety, decision-making style, thinking style, critical thinking, cognitive approach to learning, learning style, attributional style, internality-externality, stability-instability, global-specific, creativity, curiosity, playfulness, exploration, mental strength, grit, flourishing, gratitude, inspiration, spirituality, hedonism, materialism/material values, perceptions, sentiments, and/or other psychological parameters.

Achievement motivation may include compensatory effort, competitiveness, confidence in success, dominance, eagerness to learn, engagement, fearlessness, flexibility, flow, goal setting, independence, internality, persistence, preference in difficult tasks, pride in productivity, self-control, status orientation, ambition, self-assurance, and/or other psychological parameters. Motivation may include mastery, purpose, autonomy, and/or other psychological parameters.

Personality inventory may include neuroticism, openness, conscientiousness, extraversion, and agreeableness and/or other psychological parameters. Neuroticism may include anxiety, impulsiveness, vulnerability, and/or other psychological parameters. Openness may include fantasy, feelings/empathy, action, and/or other psychological parameters. Conscientiousness may include achievement striving, competence, self-discipline, and/or other psychological parameters. Extraversion may include warmth assertiveness, activity, and/or other psychological parameters. Agreeableness may include trust, altruism, modesty, and/or other psychological parameters.

Cultural values may include individualism, indulgence, long term orientation, masculinity, power distance, uncertainty avoidance, and/or other psychological parameters. Competitiveness may include avoidant, collaborative, competitive affectivity, dependent, dominant, general competitiveness, independent, personal enhancement, and/or other psychological parameters.

Positive and negative affect before, during, and/or after engaging in the online game may include hostility, joviality, negative emotions, positive emotions, sadness, self-assurance, and/or other psychological parameters. Communication style may include feeler, intuitor, sensor, thinker, and/or other psychological parameters.

Wellbeing may include social wellbeing, psychological wellbeing, physical wellbeing, physical activity, sleep, bounded reciprocity, resilience grit, and/or other psychological parameters.

Personality may include anger, hostility, depression, self-conscientiousness, excitement-seeking, positive emotions, gregariousness, ideas, values, aesthetics, tender-mindedness, straightforwardness, compliance, deliberation, order, dutifulness, and/or other psychological parameters.

Emotional style may include resilience, outlook, social intuition, self-awareness, sensitivity to context, attention, and/or other psychological parameters.

Goal orientation may include mastery approach/learning goal orientation, performance approach/performance goal orientation, performance avoid/avoidance goal orientation, and/or other psychological parameters.

Work-related quality of life may include structure, boundaries, focus, efficiency, information provision, communication, psychological support, stress at/from work, psychological safety, connectedness with team, motivation to work, adaptability, job/career satisfaction, control at work, home-work interface, general wellbeing, working conditions, and/or other psychological parameters.

Mindfulness may include observing, describing, acting with awareness, non-judgment, non-reactivity, and/or other psychological parameters.

Emotional intelligence may include emotion perception, emotion expression, emotion management, emotion regulation, impulse control, relationships, stress management, and/or other psychological parameters.

Social connectedness may include social connectedness, loneliness, membership self-esteem, private self-esteem, public self-esteem identity self-esteem, interdependent self, independent self, social avoidance, social distress, and/or other psychological parameters.

Decision-making style may include respected, confident, spontaneous, dependent, vigilant, avoidant, brooding, intuitive, anxious, and/or other psychological parameters.

Thinking style may include intuitive, experiential, analytical, rational, and/or other psychological parameters.

Cognitive approaches to learning may include avoidant, participative, competitive, collaborative, dependent, independent, and/or other psychological parameters.

Learning style may include visual (spatial), aural (auditory-musical), verbal (linguistic), physical (kinesthetic), logical (mathematical), social (interpersonal), solitary (intrapersonal), and/or other psychological parameters.

Mental strength may include tenacity, confidence, optimism, adaptability, self-awareness, reliability, responsibility, well-being, and/or other psychological parameters.

Flourishing may include positive emotion, engagement, relationships, meaning, accomplishment, health, loneliness, and/or other psychological parameters.

The parameter values of the psychological parameters may be a number score on a predetermined range unique to each psychological parameter, a letter score, and/or other type of value than may characterize intensity of the psychological parameter that the psychological parameter value defines and thus a particular subject as whole. The intensity may represent significance of a given psychological parameter that of the particular subject is.

Interface presentation component 110 may be configured to effectuate presentation of outcome interface 112. Outcome interface 112 may be presented via client computing platforms 104 associated with the subjects. Outcome interface 112 may enable selection and/or entry of one or more psychological outcomes from a catalog of psychological outcomes by individual ones of the subjects. In some implementations, the catalog may be unmodifiable. In some implementations, the catalog may be modifiable by an administrator, caregivers, and/or others to add or remove psychological outcomes. In some implementations, outcome interface 112 may include one or more subject interface elements. The subject interface elements may be configured to facilitate subject entry and/or selection. An individual subject interface element may represent an individual psychological outcome and be configured to be selected by the subjects. By way of non-limiting illustration, the subject interface elements may include one or more of text input fields, drop down menus, check boxes, virtual buttons, and/or other subject interface elements. For example, a text input field may facilitate searching for a psychological outcome in the catalog. For example, the text input field may facilitate entering a psychological outcome not included in the catalog. In some implementations, the selected and/or entered psychological outcomes by the subjects may be included in the respective psychological profiles associated with the subjects.

A given psychological outcome may be associated with changes in parameter value for one or more psychological parameters that correspond to the given psychological outcome. As such, the given psychological outcome may be achieved by achieving the associated changes in parameter value for the corresponding one or more psychological parameters. In some implementations, content identifying component 114 may be configured to determine the one or more psychological parameters that correspond to and facilitate achievement of the psychological outcomes selected/entered. By way of non-limiting illustration, the psychological outcomes may include a first psychological outcome and a second psychological outcome. The first psychological outcome may be associated with changes in parameter value for a first psychological parameter and a second psychological parameter (different from the first psychological parameter). The second psychological outcome may be associated with changes in parameter value for a third psychological parameter. The first subject may select the first psychological outcome and the second psychological outcome via outcome interface 112 to achieve.

Changes in the parameter value for the one or more psychological parameters may include a change in percentage or a particular amount of points, score, or other type of value in each of the parameter values that the psychological outcome corresponds to (e.g., the parameter value for each of the first psychological parameter and the second psychological parameter increase by 10%); a collective change in percentage or a collective change in a particular amount of points, score, or other type of value in the parameter values that the psychological outcome corresponds to (e.g., the parameter values for the first psychological parameter and the second psychological parameter decrease by 30 points collectively); a change in percentage or a particular amount of point, score, or other type of value within a particular amount of time (i.e., a particular rate of change); the parameter value to a given psychological parameter reaching a particular threshold; the parameter value to the given psychological parameter reaching the particular threshold at a particular time or within a particular amount of time; and/or other changes in the parameter value for the one or more psychological parameters. The change in the percentage or particular amount of points, score, or other type of value may include an increase or decrease of such. The particular threshold may be predetermined and correspond with a given psychological parameter and/or modifiable by caregivers. It should be understood that the changes parameter values to the psychological parameters described herein are not intended to be limiting and parameter values to psychological parameters may change in a plurality of ways not explicitly listed herein.

Content identifying component 114 may be configured to receive indication of the psychological outcomes selected and/or entered by the subjects. The indication may be received from client computing platforms 104. The indication may be received via a network. Content identifying component 114 may be configured to identify therapeutic content from content that individual online platforms 116 present. The therapeutic content may be identified based on the subject information, the psychological outcomes, the associated psychological parameters, and/or other information. In some implementations, content identifying component 114 may be configured to cause the therapeutic content to be generated for the individual online platforms 116. In some implementations, the therapeutic content may be generated based on the content currently presented by the individual online platforms 116, content not yet presented by individual online platforms 116, other content associated with individual online platforms 116, content not associated with individual online platforms 116, and/or other content.

In some implementations, content identifying component 114 may be configured to transmit the identified therapeutic content to individual online platforms 116 for presentation by online platforms 116 to the subjects. For example, transmission of the identified therapeutic content may be via the network to online platforms 116. The therapeutic content may be transmitted to the online platforms 116 according to a schedule (e.g., daily at 12 PM), immediately subsequent to identifying the therapeutic content, upon selection (e.g., by an administrator of system 100) of a virtual button to transmit, and/or at other times or upon other events. In some implementations, content identifying component 114 may be configured to effectuate presentation of the therapeutic content via online platforms 116. The therapeutic content may be presented to the subjects via individual online platforms 116 and facilitate achievement of the psychological outcomes. That is, the therapeutic content may facilitate changes in parameter value to the psychological parameter(s) that correspond to the psychological outcomes.

By way of non-limiting illustration, first therapeutic content may be identified and presented to the first subject via online platforms 116. The first therapeutic content may facilitate changes in parameter value to the first psychological parameter and the second psychological parameter that correspond to the first psychological outcome.

Profile update component 118 may be configured to receive interaction information from individual online platforms 116. The interaction information may characterize engagement by the subjects with the therapeutic content. In some implementations, the interaction information may be received in an ongoing manner. The term “ongoing manner”, as used herein, may refer to continuing to perform an action (e.g., update) periodically (e.g., every 30 seconds, every minute, every hour, etc.) until receipt of an indication to terminate. The indication to terminate may include a selection of a virtual button to cease the action, and/or other indications of termination. In some implementations, the indication to terminate may include a predefined time (e.g., a date and time). The predefined time may be defined by an administrator of system 100, a caregiver, a subject associated with the psychological profile being updated, and/or other subject. Caregivers of the subjects, as used herein, may include one or more of a doctor, a therapist, a psychiatrist, a mentor, a specialist (e.g., addiction specialist), and/or other caregivers. In some implementations, individual online platforms 116 may define when the interaction information is received by profile update component 118.

The engagement by the individual subjects may define behavior patterns of the individual subjects with or based on the therapeutic content. The behavior patterns of the individual subjects may include individual actions, sets of actions, ordered sets of actions, time spent by the individual subjects engaging with the therapeutic content and/or the online platforms, spending patterns of the subjects, completed tasks by the individual subjects, uncompletion tasks by the individual subjects, failure of tasks by the individual subjects, and/or other behavior patterns. In some implementations, the behavior patterns may include multiple of the individual actions, the sets of actions, and the ordered set of actions. The actions may include one or more of a purchase based on the therapeutic content, a sale, a trade, a donation, a subject selection of the content, gameplay (e.g., mini-game, battle, competition, etc.) based on the therapeutic content, communication of the individual subjects with other particular subjects, completion of tasks by the subjects, frequent interaction with the content, formation of alliances by the subjects, and/or other actions. The spending patterns may indicate an amount of currency (e.g., real-world money, virtual money, points, etc.) spend, an amount of currency earned, an amount of currency donated, and/or other indications. By way of non-limiting illustration, first interaction information may be received that characterizes engagement by the first subject with the first therapeutic content.

Profile update component 118 may be configured to update the psychological profiles of the individual subjects. Updating the psychological profiles (i.e., the parameter values to the psychological parameters) may be based on the interaction information of the individual subjects and/or other information. The psychological profiles may be updated in an ongoing manner. The psychological profiles of the subjects may include the behavior patterns of the subjects. In some implementations, updating the psychological profiles may include updating the behavior patterns ongoingly based on the interaction information.

Progress component 120 may be configured to determine a progress metric value to a progress metric. The progress metric value may be determined in an ongoing manner. Determining the progress metric value may be based on the interaction information, the psychological profiles, both, and/or other information. Thus, individual subjects may be associated with an individual one of the progress metrics, and values thereof. In some implementations, the progress metrics and values thereof may be stored as or otherwise included in the subject information of the respective subjects. The progress metric value of the progress metric may indicate progress towards achieving the psychological outcomes selected. That is, the progress metric value may indicate progress towards changes in parameter value for the one or more psychological parameters that correspond to the psychological outcomes selected (or entered). By way of non-limiting example, the progress metric value may be out of a total progress value (e.g., 75% progress out of 100%, 20 out of 25, etc.). In some implementations, the progress metric values may be presented to the respective subjects via client computing platforms 104. The progress metrics and values thereof may be presented as the progress metric value out of the total progress value, a progress bar, a progress animation, and/or other presentations.

By way of non-limiting illustration, a progress metric value to a first progress metric may indicate progress of the first subject towards achieving the first psychological outcome, i.e., changes in parameter value of the first and second psychological parameters. The progress metric value may be determined based on the first interaction information, the first psychological profile (newly updated), and/or other information.

Progress component 120 may be configured to adjust the therapeutic content. The therapeutic content may be adjusted based on the progress metric value and/or other information. The therapeutic content may be adjusted in an ongoing manner. Adjusting the therapeutic content may facilitate subsequently achieving changes in parameter value for one or more psychological parameters that correspond to another one of the psychological outcomes selected or entered by the subjects. In some implementations, adjusting the therapeutic content may facilitate disengaging the subjects from therapeutic content presented to engaged with other therapeutic content. In some implementations, adjusting the therapeutic content may be performed automatically and/or based on machine learning by progress component 120. The machine learning may be based on known techniques. In some implementations, adjusting the therapeutic content may be further based on caregiver input and/or subject input. The subject input may indicate that the subject has achieved the psychological outcomes selected/entered. By way of non-limiting illustration, the first therapeutic content may be adjusted to facilitate achieving changes in parameter value for the third psychological parameter (i.e., the second psychological outcome) subsequent to the first psychological outcome.

In some implementations, adjusting the therapeutic content may include causing content identifying component 114 to identify new therapeutic content for the other psychological outcome(s) with the same techniques previously utilized to determine the therapeutic content. In some implementations, adjusting the therapeutic content may include adding content of individual online platforms 116, removing content of individual online platforms 116, changing features of the therapeutic content, and/or other adjustments. By way of non-limiting example, the features of the therapeutic content that may be changed may include one or more of a color, a font, an undertone of messages included in the therapeutic content (e.g., calming, soothing, direct/frank, etc.), and/or other features.

In some implementations, the therapeutic content may be gradually adjusted in accordance with timing information. The timing information may define an amount of time for the subject to achieve the one or more psychological outcomes collectively and/or individually. For example, the therapeutic content may be gradually adjusted so that a given psychological outcome is achieved within 6 months. In some implementations, the timing information may be predetermined, determined based on caregiver input, selected by the subjects, or entered by the subjects. In some implementations, the timing information may be predetermined based on the psychological outcome(s) selected/entered. For example, a psychological outcome selected may be calmness/decrease anxiety for achievement in 8 months. In some implementations, the predetermined timing information may be modified based on the caregiver input. In some implementations, the subjects may modify the timing information (e.g., the 8 months) to a longer amount of time or a shorter amount of time. In some implementations, the predetermined timing information may have minimum and/or maximum parameters that the timing information may not be modified past. For example, the calmness/decrease anxiety may be achieved in a minimum of 6 months and no shorter. The adjusted therapeutic content may be transmitted by content identifying component 114 to online platforms 116 for presentation. In some implementations, content identifying component 114 may be configured to effectuate presentation of the adjusted therapeutic content via online platforms 116.

Progress component 120 may be configured to receive the caregiver input. The caregiver input may be received via client computing platforms 104 associated with caregivers. In some implementations, the caregiver input may characterize the change in the parameter value to the one or more psychological parameters (corresponding to the psychological outcomes selected). In some implementations, the caregiver input may include notes about and associated with the changes in the parameter value. Thus, adjusting the therapeutic content (by progress component 120) may be based on the caregiver input. By way of non-limiting illustration, the caregiver input may include a second progress metric value. The second progress metric value may update the progress metric value to the first progress metric. In some implementations, the caregiver input may include and define the amount of time for the subject and/or modify the amount of time for the subject to achieve the psychological outcomes selected (i.e., the timing information).

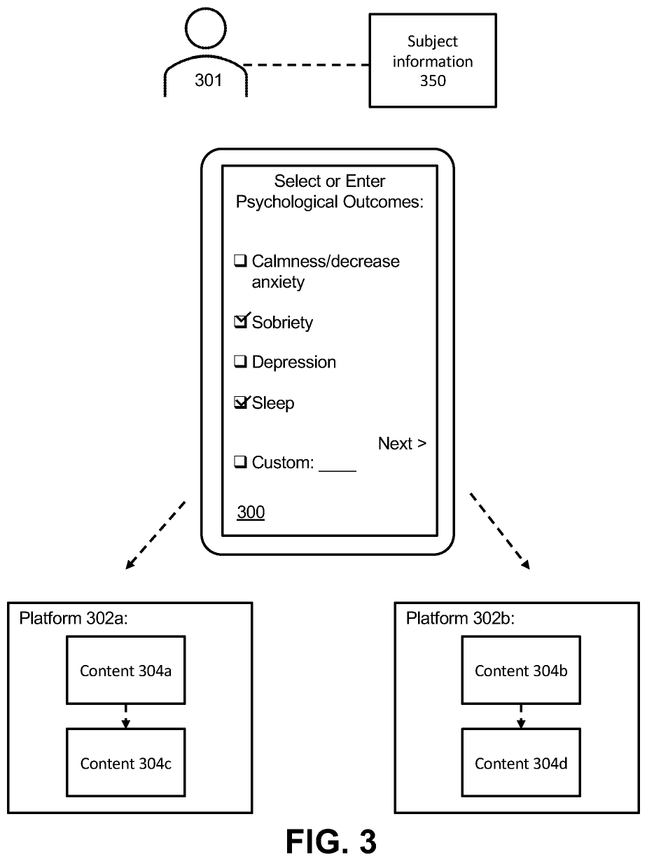

FIG. 3 illustrates an example implementation, in accordance with one or more implementations. FIG. 3 illustrates a subject 301 who is associated with subject information 350. Subject information 350 may be received (i.e., by system 100 of FIG. 1) and may include a psychological profile for subject 301. Subject 301 may be presented an outcome interface 300 that enables subject 301 to selected and/or enter psychological outcomes to achieve. Subject 301 may selected sobriety and sleep. Based on selecting sobriety, a therapeutic content 304a may be identified for a platform 302a to present for subject 301 and a therapeutic content 304b may be identified for a platform 302b to present for subject 301. Based on interaction information received from platform 302a and platform 302b that characterizes engagement by subject 301 with therapeutic content 304a and 304b, respectively, therapeutic content 304c may be identified for platform 302a and therapeutic content 304d may be identified for platform 302b to present on the respective platforms. Therapeutic content 304c and 304d may be identified based on a progress metric value to a progress metric (not pictured) and subject information 350 (e.g., the psychological profile). The progress metric value may be determined based on the interaction information. Therapeutic content 304c and 304d may facilitate achieving sleep instead of the sobriety.

In some implementations, server(s) 102, client computing platform(s) 104, and/or external resources 128 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which server(s) 102, client computing platform(s) 104, and/or external resources 128 may be operatively linked via some other communication media.

A given client computing platform 104 may include one or more processors configured to execute computer program components. The computer program components may be configured to enable an expert or subject associated with the given client computing platform 104 to interface with system 100 and/or external resources 128, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

External resources 128 may include sources of information outside of system 100, external entities participating with system 100, and/or other resources. In some implementations, some or all of the functionality attributed herein to external resources 128 may be provided by resources included in system 100.

Server(s) 102 may include electronic storage 130, one or more processors 132, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 130 may comprise non-transitory storage media that electronically stores information. The electronic storage media of electronic storage 130 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 130 may include one or more virtual storage resources (e.g., cloud storage, a virtual private network, and/or other virtual storage resources). Electronic storage 130 may store software algorithms, information determined by processor(s) 132, information received from server(s) 102, information received from client computing platform(s) 104, and/or other information that enables server(s) 102 to function as described herein.

Processor(s) 132 may be configured to provide information processing capabilities in server(s) 102. As such, processor(s) 132 may include one or more of a digital processor, an analog processor, a digital circuit designed to process information, an analog circuit designed to process information, a state machine, and/or other mechanisms for electronically processing information. Although processor(s) 132 is shown in FIG. 1 as a single entity, this is for illustrative purposes only. In some implementations, processor(s) 132 may include a plurality of processing units. These processing units may be physically located within the same device, or processor(s) 132 may represent processing functionality of a plurality of devices operating in coordination. Processor(s) 132 may be configured to execute components 110, 114, 118, and/or 120, and/or other components. Processor(s) 132 may be configured to execute components 110, 114, 118, and/or 120, and/or other components by software; hardware; firmware; some combination of software, hardware, and/or firmware; and/or other mechanisms for configuring processing capabilities on processor(s) 132. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 110, 114, 118, and/or 120 are illustrated in FIG. 1 as being implemented within a single processing unit, in implementations in which processor(s) 132 includes multiple processing units, one or more of components 110, 114, 118, and/or 120 may be implemented remotely from the other components. The description of the functionality provided by the different components 110, 114, 118, and/or 120 described below is for illustrative purposes, and is not intended to be limiting, as any of components 110, 114, 118, and/or 120 may provide more or less functionality than is described. For example, one or more of components 110, 114, 118, and/or 120 may be eliminated, and some or all of its functionality may be provided by other ones of components 110, 114, 118, and/or 120. As another example, processor(s) 132 may be configured to execute one or more additional components that may perform some or all of the functionality attributed below to one of components 110, 114, 118, and/or 120.

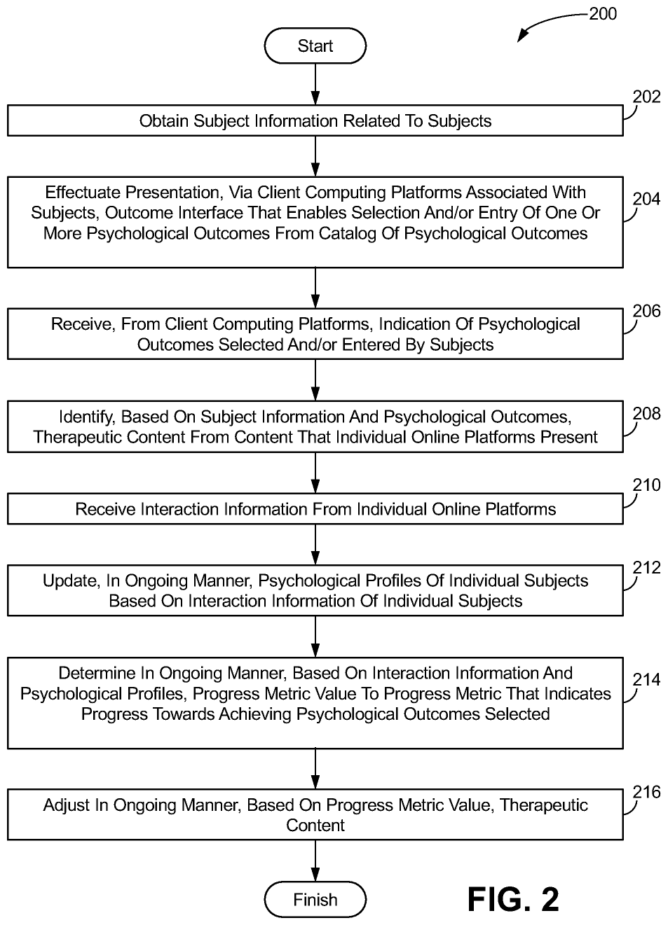

FIG. 2 illustrates a method 200 to provide a digital experience adapted based on a subject selection to effect particular attributes, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include obtaining subject information related to subjects. The subject information may characterize psychological profiles of the individual subjects. The psychological profile may be defined by parameter values of psychological parameters. Operation 202 may be performed by one or more hardware processors configured by machine-readable instructions including a component that is the same as or similar to content identifying component 114, in accordance with one or more implementations.

An operation 204 may include effectuate presentation an outcome interface that enables selection and/or entry of one or more psychological outcomes from a catalog of psychological outcomes. A given psychological outcome may be associated with changes in parameter value for one or more psychological parameters that correspond to the given psychological outcome. Operation 204 may be performed by one or more hardware processors configured by machine-readable instructions including a component that is the same as or similar to interface presentation component 110, in accordance with one or more implementations.

An operation 206 may include receiving, from the client computing platforms, indication of the psychological outcomes selected and/or entered by the subjects. Operation 206 may be performed by one or more hardware processors configured by machine-readable instructions including a component that is the same as or similar to content identifying component 114, in accordance with one or more implementations.

An operation 208 may include identifying, based on the subject information and the one or more psychological outcomes, therapeutic content from content that individual online platforms present. The therapeutic content may be presented to the subjects via the individual online platforms and facilitate achievement of the one or more psychological outcomes. Operation 208 may be performed by one or more hardware processors configured by machine-readable instructions including a component that is the same as or similar to content identifying component 114, in accordance with one or more implementations.

An operation 210 may include receiving interaction information from the individual online platforms. The interaction information may characterize engagement by the subjects with the therapeutic content. Operation 210 may be performed by one or more hardware processors configured by machine-readable instructions including a component that is the same as or similar to profile update component 118, in accordance with one or more implementations.

An operation 212 may include updating in an ongoing manner the psychological profiles of the individual subjects based on the interaction information of the individual subjects. Operation 212 may be performed by one or more hardware processors configured by machine-readable instructions including a component that is the same as or similar to profile update component 118, in accordance with one or more implementations.

An operation 214 may include determining in an ongoing manner, based on the interaction information and the psychological profiles, a progress metric value to a progress metric that indicates progress towards achieving the psychological outcomes selected. Operation 214 may be performed by one or more hardware processors configured by machine-readable instructions including a component that is the same as or similar to progress component 120, in accordance with one or more implementations.

An operation 216 may include adjusting in an ongoing manner, based on the progress metric value, the therapeutic content. Operation 216 may be performed by one or more hardware processors configured by machine-readable instructions including a component that is the same as or similar to progress component 120, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A system configured to provide a digital experience adapted based on a subject selection to effect particular attributes, the system comprising:
one or more processors configured by machine-readable instructions to:
obtain subject information related to subjects, wherein the subject information characterizes psychological profiles of the individual subjects, wherein the psychological profile is defined by parameter values of psychological parameters;
effectuate presentation, via client computing platforms associated with the subjects, of an outcome interface comprising subject interface elements that enable selection and/or entry of one or more psychological outcomes from a catalog of psychological outcomes, wherein the subject interface elements include at least one or more of a checkbox, a text input field, and a virtual button, wherein a given psychological outcome is associated with changes in parameter value for one or more psychological parameters that correspond to the given psychological outcome such that the given psychological outcome is achieved by achieving the associated changes in parameter value for the corresponding one or more psychological parameters;
receive, from the client computing platforms, indication of the psychological outcomes selected and/or entered by the subjects via the subject interface elements;
identify, based on the subject information and the psychological outcomes, preliminary therapeutic content from content that individual online platforms present;
generate secondary therapeutic content based on the identified preliminary therapeutic content;
adjust the content presented by the individual online platforms by effectuating presentation of the secondary therapeutic content to the subjects via the individual online platforms and removing other content to facilitate achievement of the psychological outcomes;
receive interaction information from the individual online platforms, wherein the interaction information characterizes engagement by the subjects with the secondary therapeutic content;
update in an ongoing manner the psychological profiles of the individual subjects based on the interaction information of the individual subjects;
determine in an ongoing manner, based on the interaction information and the psychological profiles, a progress metric value to a progress metric that indicates progress towards achieving the psychological outcomes selected; and
adjust in an ongoing manner, based on the progress metric value, the secondary therapeutic content.

2. The system of claim 1, wherein the one or more processors are configured by the machine-readable instructions to:
transmit the secondary therapeutic content to the individual online platforms for presentation by the online platforms to the subjects.

3. The system of claim 1, wherein the psychological outcomes include a first psychological outcome and a second psychological outcome, wherein the therapeutic content facilitates achieving changes in parameter value for one or more psychological parameters that correspond to the first psychological outcome, wherein adjusting the therapeutic content facilitates subsequently achieving changes in parameter value for one or more psychological parameters that correspond to the second psychological outcome.

4. The system of claim 1, wherein adjusting the secondary therapeutic content is performed automatically and based on machine learning.

5. The system of claim 1, wherein the one or more processors are configured by the machine-readable instructions to:
receive, via a client computing platform associated with a caregiver, caregiver input, wherein the caregiver input characterizes the change in the parameter value to the one or more psychological outcomes, wherein adjusting the secondary therapeutic content is based on the caregiver input.

6. The system of claim 5, wherein the caregiver input includes a second progress metric value, wherein the second progress metric value updates the progress metric value.

7. The system of claim 1, wherein the secondary therapeutic content is gradually adjusted in accordance with timing information, wherein the timing information defines an amount of time for the subject to achieve the one or more psychological outcomes.

8. The system of claim 7, wherein the timing information is predetermined or determined based on caregiver input, wherein the caregiver input defines the amount of time for the subject and/or modifies the amount of time for the subject.

9. A method to provide a digital experience adapted based on a subject selection to effect particular attributes, the method comprising:
obtaining subject information related to subjects, wherein the subject information characterizes psychological profiles of the individual subjects, wherein the psychological profile is defined by parameter values of psychological parameters;
effectuating presentation, via client computing platforms associated with the subjects, of an outcome interface comprising subject interface elements that enable selection and/or entry of one or more psychological outcomes from a catalog of psychological outcomes, wherein the subject interface elements include at least one or more of a checkbox, a text input field, and a virtual button, wherein a given psychological outcome is associated with changes in parameter value for one or more psychological parameters that correspond to the given psychological outcome such that the given psychological outcome is achieved by achieving the associated changes in parameter value for the corresponding one or more psychological parameters;
receiving, from the client computing platforms, indication of the psychological outcomes selected and/or entered by the subjects via the subject interface elements;
identifying, based on the subject information and the psychological outcomes, preliminary therapeutic content from content that individual online platforms present;
generate secondary therapeutic content based on the identified preliminary therapeutic content;
adjusting the content presented by the individual online platforms by effectuating presentation of the secondary therapeutic content the subjects via the individual online platforms and removing other content to facilitate achievement of the psychological outcomes;
receiving interaction information from the individual online platforms, wherein the interaction information characterizes engagement by the subjects with the secondary therapeutic content;
updating in an ongoing manner the psychological profiles of the individual subjects based on the interaction information of the individual subjects;
determining in an ongoing manner, based on the interaction information and the psychological profiles, a progress metric value to a progress metric that indicates progress towards achieving the psychological outcomes selected; and
adjusting in an ongoing manner, based on the progress metric value, the therapeutic content.

10. The method of claim 9, further comprising:
transmitting the secondary therapeutic content to the individual online platforms for presentation by the online platforms to the subjects.

11. The method of claim 9, wherein the psychological outcomes include a first psychological outcome and a second psychological outcome, wherein the therapeutic content facilitates achieving changes in parameter value for one or more psychological parameters that correspond to the first psychological outcome, wherein adjusting the therapeutic content facilitates subsequently achieving changes in parameter value for one or more psychological parameters that correspond to the second psychological outcome.

12. The method of claim 9, wherein adjusting the secondary therapeutic content is performed automatically and based on machine learning.

13. The method of claim 9, further comprising:
receiving, via a client computing platform associated with a caregiver, caregiver input, wherein the caregiver input characterizes the change in the parameter value to the one or more psychological outcomes, wherein adjusting the secondary therapeutic content is based on the caregiver input.

14. The method of claim 13, wherein the caregiver input includes a second progress metric value, wherein the second progress metric value updates the progress metric value.

15. The method of claim 9, wherein the secondary therapeutic content is gradually adjusted in accordance with timing information, wherein the timing information defines an amount of time for the subject to achieve the one or more psychological outcomes.

16. The method of claim 15, wherein the timing information is predetermined or determined based on caregiver input, wherein the caregiver input defines the amount of time for the subject and/or modifies the amount of time for the subject.
